(19) United States
(12) Patent Application Publication
(10) Pub. No.: US 2003/0114820 A1
(43) Pub. Date: Jun. 19, 2003

(54) Fluid containment textile structure

(76) Inventor: Martin Wildeman, Spartanburg, SC (US)

Correspondence Address: JAMES M. ROBERTSON, J.M. ROBERTSON INTELLECTUAL, PROPERTY SERVICES, LLC, 233 SOUTH PINE STREET, SPARTANBURG, SC, 29302, US

(21) Appl. No.: 10/015,481

(22) Filed: Dec. 13, 2001

(51) Int. Cl. D01D 1/00; A61F 13/15; A61F 13/20

(52) U.S. Cl. 604/383; 28/140

(57) ABSTRACT

A method for forming a launderable fluid containment textile structure composite of stitch bonded construction such as is useful in an incontinence pad or diaper and a textile composite formed by such method. The textile composite includes a fluid retention layer of non-woven batting formed from a plurality of intermingled textile fibers. A plurality of bonding yarns extend in a repeating stitch bonding pattern through the fluid retention layer such that the bonding yarns form a user contact surface over the fluid retention layer. The stitch bonding pattern is characterized by a relatively low stitch density. Enhanced bulkiness and dimensional stability against laundering induced shrinkage is imparted by introduction of mechanically induced compressive shrinkage.

### TECHNICAL FIELD

The present invention is directed generally to a textile of stitch bonded construction and more particularly to a mechanically compressed textile of stitch bonded construction useful in a reusable launderable absorbent structure.

### BACKGROUND OF THE INVENTION

It is generally known to utilize a reusable absorbent pad referred to as an incontinence pad as part of the bedding of persons having an inability to control the discharge of bodily fluids. The purpose of such pads is to draw urine or other fluid as may be discharged away from the person thereby relieving the discomfort which may arise from extended contact with such fluids. Such pads typically incorporate one or more layers of felt material within the interior of the pad to draw the fluids away from the surface and to hold such fluids in place until the pad may be laundered. Through such absorption and retention by the fluid retaining layer, the surface of the pad is maintained in a relatively dry state. A fluid impermeable backing is typically disposed beneath the felt to prevent leakage of the retained fluid onto the underlying bed linens. The same constructions may also be formed into reusable diapers to be worn by a user.

Historically, structures forming incontinence pads and reusable diapers have been manufactured according to a substantially stepwise procedure wherein the fabric forming the user contact surface is formed separately from the material forming the fluid retention layer and a quilting process is thereafter applied to hold those layers together. Such a manufacturing process has been found to be potentially cost prohibitive.

### SUMMARY OF THE INVENTION

The present invention provides alternatives and advantages over the prior art by providing a launderable fluid containment textile composite suitable for use in an incontinence pad or reusable diaper wherein the outer surface layer of the composite which contacts the user is formed directly over a fluid retaining non-woven batting of blended hydrophobic and hydrophilic fibers by use of a highly efficient stitch bonding procedure which simultaneously stabilizes the composite and forms the user contact surface. The formed composite may thereafter undergo mechanically induced shrinkage to introduce compression in at least the length direction to thereby enhance resistance to laundering induced shrinkage and to increase bulkiness.

The user contact surface which is made up of the cooperating stitch elements applied during the stitch bonding operation serves to cover the underlying non-woven batting layer and provides the desired soft feel which is generally desired for use in a bedding or diaper environment.

The selection of stitching yarns and the pattern utilized for application of the stitches forming the user contact surface is such that the desired level of surface coverage may be obtained while nonetheless operating the stitch application equipment at an extremely high throughput rate at a relatively low number of stitches per inch such that the stitch bonding equipment may be operated at extremely high efficiency. Coverage characteristics may be enhanced by subjecting the resulting composite to mechanically induced elongate compression to add bulk to the structure while improving long term dimensional stability against substantial shrinkage during subsequent laundering operations.

The stitch bonded composite formed according to the present invention may be utilized as a component of an incontinence pad or in such other applications such as a diaper (including adult protective garments) wherein the resultant cover and absorptive characteristics are useful.

According to one aspect of the invention a fluid containment textile composite of highly efficient and simple stitch bonded construction is provided.

According to a further aspect of present invention a fluid containment textile composite of stitch bonded construction which has undergone an effective degree of mechanically induced shrinkage in the length dimension and thickening in the height dimension is provided.

According to a further aspect of the present invention the textile composite may be formed of an interior non-woven batting layer of blended hydrophobic polyester and hydrophilic rayon fiber constituents stabilized by stitchbonding yarns extending through the non-woven batting layer and forming a user contact surface directly over the non-woven batting layer. The stitch bonding yarns may be open end or ring spun yarns including polyester and cotton or other cellulosic fiber constituents such as rayon and the like.

According to an additional aspect of the present invention, the stitch bonding yarns forming the user contact surface of the textile composite may be applied through the non-woven batting layer according to a repeating stitch configuration such that the stitch bonding pattern is characterized by a low stitch density in the machine direction of the formed composite and the textile composite is mechanically compressed to induce between about 5 percent and about 20 percent shrinkage in the longitudinal direction thereby providing further enhanced surface coverage at low stitch density which may be maintained through multiple washing cycles.

According to yet a further aspect of the present invention, the stitch bonding yarns forming the user contact surface of the textile composite may be applied through the non-woven layer according to a repeating stitch configuration such that the stitch bonding pattern is characterized by a relatively low stitch density (i.e. long stitches) in the machine direction of the formed composite and the textile composite is mechanically compressed to induce a substantial increase in thickness thereby providing enhanced bulkiness at low stitch density which may be maintained through multiple washing cycles.

### BRIEF DESCRIPTION OF THE DRAWINGS

The principles of the present invention are set forth in the following detailed description through reference to the accompanying drawings which are incorporated in and which constitute a part of this specification and in which:

FIG. 1 illustrates a fluid containment composite structure in the form of a pad as may incorporate a fluid containment textile composite according to the present invention including an enlarged view of the user contact surface of such fluid containment textile composite;

FIG. 1A illustrates a diaper structure as may be formed from the fluid containment composite structure illustrated in FIG. 1;

FIG. 2 is a cross-sectional view of a potentially preferred embodiment of a fluid containment textile composite construction; and

FIG. 3 is a point diagram of a potentially preferred stitch bonding pattern as may be utilized in the construction of a fluid containment textile composite.

While the invention has been illustrated and generally described above, and will hereafter be described in detail in connection with certain potentially preferred embodiments, it is to be appreciated that the foregoing general description as well as the particularly illustrated and described embodiments as may be set forth herein are exemplary and explanatory only. Accordingly, there is no intention to limit the invention to such particularly illustrated and described embodiments. On the contrary, it is intended that the present invention shall extend to all alternatives, modifications, and equivalents as may embody the broad principles of the invention within the full spirit and scope thereof.

### DESCRIPTION OF THE PREFERRED EMBODIMENT

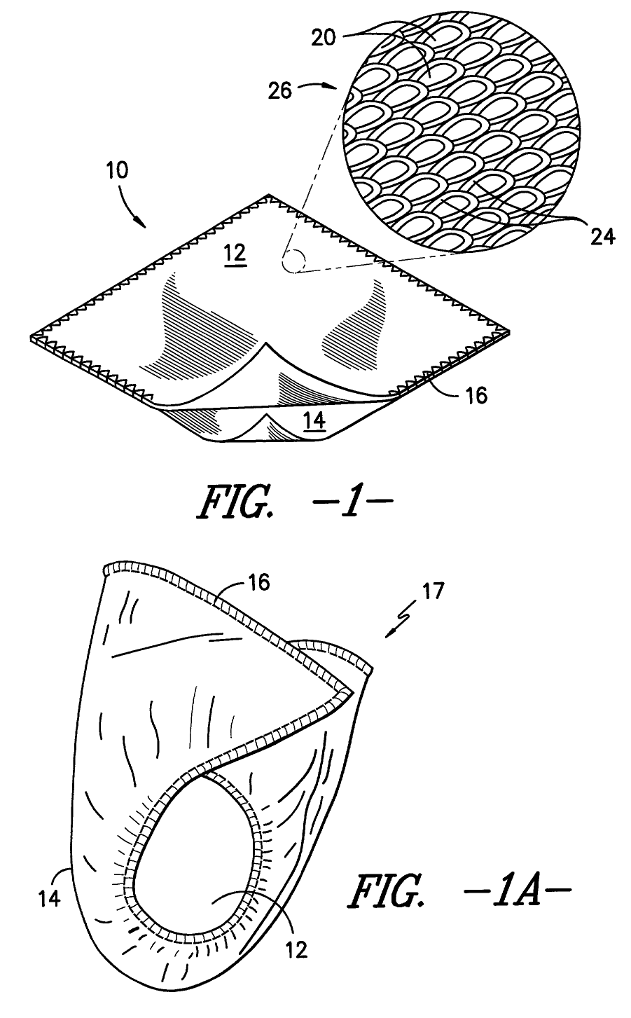

In general, the present invention is directed to an improved fluid containment textile. In FIG. 1, there is illustrated an incontinence pad 10 such as may be used to collect and retain discharges of urine, blood, or other bodily fluids as may be released from time to time. By way of example only, such an incontinence pad 10 may find application in the bedding or wheelchairs of hospital patients who experience intermittent loss of bladder control, thereby resulting in uncontrollable discharges of urine. In such an environment, the incontinence pad 10 serves the function of collecting and retaining the discharged fluid so as to prevent extended contact between such fluid and the patient.

As illustrated, the incontinence pad 10 includes a textile composite 12 to be described further hereinafter which may be joined to an underlying fluid barrier layer 14 by seam stitching 16 or other appropriate bonding mechanisms such as adhesives or ultrasonic bonding techniques as may be known to those of skill in the art. The fluid barrier layer 14 may be any suitable liquid impermeable material including, by way of illustration only, vinyl, plastic or a coated or laminated textile structure as will be well known to those of skill in the art to which the present invention pertains. It is also contemplated that the barrier function may be carried out by a coating applied across one side of the textile composite 12. By way of example only, and not limitation, it is contemplated that such a coating may be applied by means such as knife coating, roll coating lamination and/or extrusion coating.

While the incontinence pad 10 is of a generally flat construction so as to facilitate its use as an underlay in bedding and on chairs, it is also contemplated that the textile composite 12 with adjoined fluid barrier layer 14 may be cut in a manner as will be well known to those of skill in the art and formed into a three-dimensional garment such as a diaper 17 as illustrated in FIG. 1A. In this regard, it is to be appreciated that the term “diaper” is meant to refer to any protective garment as may be worn by either a child or an adult for purposes of collecting and retaining uncontrolled discharges of bodily fluid. In such a diaper, the textile composite 12 is disposed at the interior in opposing relation to the wearer while the fluid barrier layer 14 is disposed at the exterior to effectively block fluid passage. By way of example only, and not limitation, one geometry for a diaper construction may be a generally flat hour-glass configuration foldable to a desired three-dimensional arrangement. One such diaper construction is illustrated in U.S. Pat. No. 4,516,975 to Mitchell the teachings of which are incorporated herein by reference.

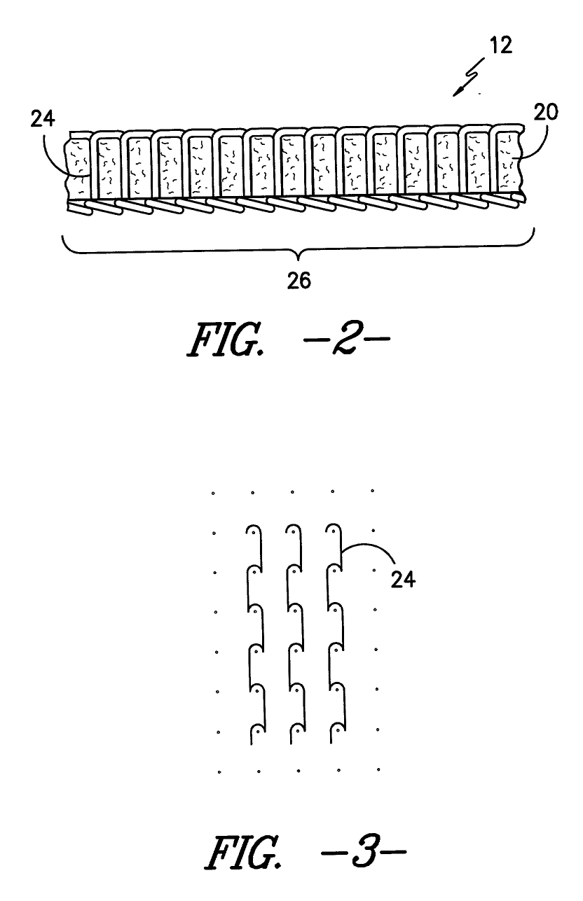

The textile composite 12 as may be used in the incontinence pad 10 and the diaper 17 is preferably a stitch bonded composite having a configuration generally as illustrated in FIG. 2. As illustrated therein, according to a potentially preferred form of the invention, the textile composite 12 includes a fluid retaining non-woven batting core 20 and a plurality of bonding yarns 24 running in a repeating pattern across the fluid retaining non-woven batting core 20 so as to provide dimensional integrity to the non-woven batting core 20 while at the same time providing a covering across the non-woven batting core 20.

In order for the textile composite portion 12 to effectively withdraw and retain fluids away from the user, the fluid retaining non-woven batting core 20 is preferably formed at least in part from a highly absorbent material. In a potentially preferred form of the invention, the material forming the fluid retaining non-woven batting core 20 is made up of a blend of staple fibers including both hydrophobic and hydrophilic constituents. The blend of material preferably includes about 10% to about 80% polyester fiber in combination with about 90% to about 20% rayon fiber. One contemplated blend of material includes about 80% polyester fiber in combination with about 20% rayon fiber.

As will be appreciated, the rayon constituent fibers are of a highly hydrophilic character thereby tending to attract fluid. Conversely, the polyester constituent fibers which surround the rayon fibers are of a highly hydrophobic character and serve to contain the fluid attracted by the rayon. According to a potentially preferred practice, both the hydrophobic and the hydrophilic fibers will be of substantially equivalent average length and denier and will be dispersed substantially uniformly throughout the fluid retaining non-woven batting core 20 such that the absorptive properties of the fluid retaining non-woven batting core 20 will be substantially uniform across its thickness. The average length of the polyester and rayon staple fibers is preferably in the range of about 2 inches to about 5 inches. The average denier of both the rayon and polyester staple fibers making up the fluid retaining non-woven batting core 20 is preferably in the range of about 3 denier to about 6 denier and is most preferably in the range of about 4 denier.

In the event that additional stability is desired within the fluid retaining non-woven batting core 20, it is contemplated that some percentage of low melt bicomponent fiber such as KOSA™ type 252 may also be included within the blend. The inclusion of such a low melt fiber permits the batting to undergo a heat fusion process wherein the temperature of the formed batting is raised to a level above the melting point of the low melt fiber thereby permitting the low melt fiber to undergo at least a partial melting so as to bind the fibers of the batting together when the temperature is reduced.

The view in FIG. 2 is taken in the cross-machine direction of the textile composite 12 as manufactured such that the so called “technical face” of the textile composite is at the bottom and the so called “technical back” is at the top. According to a potentially preferred practice, the portions of the bonding yarns 24 which extend across the technical face cooperatively define a user contact surface 26. As best seen in FIG. 1, during use the user contact surface 26 faces away from the underlying fluid barrier layer 14 (i.e. towards the user). According to the illustrated and potentially preferred embodiment, segments of the bonding yarns 24 likewise extend through the outer surface of the non-woven batting core 20 at the technical back of the textile composite 12 thereby serving to define the underside of the textile composite 12 to which the liquid impermeable barrier layer 14 is applied.

In order to promote user comfort, the user contact surface 26 is preferably of a nature such that substantial amounts of fluid will not be absorbed and retained thereat. Thus, it is contemplated that the bonding yarns 24 which cooperate to form the user contact surface 26 are preferably of a substantially hydrophobic character. However, notwithstanding the desire to maintain the user contact surface 26 in a substantially dry state, it is also desired that such user contact surface 26 be substantially nonabrasive when touched by the user. It has been found that the use of bonding yarns 24 of a spun construction which incorporate hydrophobic synthetic polymer fibers as the primary constituent in combination with cellulosic fibers as a secondary constituent imparts a soft, nonabrasive tactile character to the user contact surface while nonetheless avoiding substantial retention of fluid at the user contact surface 26 despite the substantially hydrophilic character of such cellulosic fiber.

One such spun yarn which may be particularly preferred is an open end spun yarn incorporating about 65% polyester and about 35% cotton. Of course, it is to be appreciated that a greater or lesser percentage of cotton or other cellulosic constituent such as rayon or the like may be incorporated into the bonding yarns 24 as may be desired. By way of example only, it is contemplated that the bonding yarns 24 may include as much as about 45% or more of cotton if desired. It is likewise contemplated that the cotton or other cellulosic constituent may be eliminated entirely if desired. In such a configuration, the spun character of the bonding yarns 24 nonetheless provides the desired tactile character. The use of such all polyester bonding yarns 24 may impart a brilliant white character to the user contact surface 26 which may be desirable in some applications. As will be recognized, cotton fibers may not appear to be as white as synthetic yarns. Thus, in the event that a cotton or other natural fiber constituent is utilized in the bonding yarns 24, it is contemplated that the user contact face 26 may be overdyed a shade other than white such as a light blue or the like so as to provide an aesthetically pleasing appearance. As will be appreciated, dyeing of the technical back of the textile composite may not be required since it remains substantially hidden during use.

The user contact surface 26 which is formed by the cooperating stitches of the bonding yarns 24 preferably provides substantial coverage over the non-woven batting core 20. According to a potentially preferred practice, the bonding yarns 24 are passed through the fluid retaining non-woven batting core 20 in a repeating stitch configuration using a LIBA™ type stitch bonding machine although Maliwatt or other stitch bonding equipment as may be known to those of skill in the art may also be utilized. A single bar stitch system is potentially preferred, although it is contemplated that stitch systems incorporating two or more bars may also be incorporated.

One potentially preferred arrangement of the stitches formed by the bonding yarns 24 is a chain stitch configuration as illustrated in the point diagram of FIG. 3 having a stitch notation of 1,0/0,1. If desired, the stitches may be passed over a pile sinker so as to permit the bonding yarns 24 to appear relaxed upon formation.

The stitches formed by the bonding yarns 24 are preferably applied in the cross machine direction according to about an 8 gauge to about a 20 gauge construction and will most preferably be applied according to about an 18 gauge construction, although it is contemplated that greater or lesser constructions may be utilized as desired. The stitch density in the machine direction is preferably in the range of about 4 stitches per inch to about 20 stitches per inch, and more preferably will be in the range of about 6 stitches per inch to about 16 stitches per inch, and will most preferably be in the range of about 8 stitches per inch.

As indicated previously, the bonding yarns 24 are preferably of an open end spun construction which is believed to provide a particularly soft feel to the user contact surface 20 due to the generally fuzzy character of the surface in such yarns. According to a potentially preferred practice, such spun yarns will be of a singles configuration characterized by a cotton count of about 6 to 36 and will most preferably be a 10 singles open end spun yarn.

Although an open end spun construction is potentially preferred for the bonding yarns 24, it is also contemplated that yarns of differing constructions may likewise be utilized. By way of example only, and not limitation, alternative yarns may include other spun constructions such as ring spun yarns as well as textured filament yarns such as textured polyester yarn and the like as may be known to those of skill in the art. Such textured polyester yarn will preferably be characterized by a linear density of about 200 to about 400 denier and will most preferably have a linear density of about 300 denier. It is further contemplated that the bonding yarns 24 may be treated with an antimicrobial agent as may be known to those of skill in the art so as to further promote the sanitary character of the composite 12.

As indicated, according to a potentially preferred practice, the user contact surface 26 is formed across the technical face of the textile compositely. It has been found that the application of the described stitch construction across the textile composite 12 yields an arrangement of cooperating stitch elements across the technical face substantially as illustrated in FIG. 1. As will be appreciated, while the voids surrounding the yarn segments forming the individual stitch elements have been greatly enhanced for illustrative purposes, in actual practice the stitch elements formed by the individual bonding yarns 24 serve to provide substantial coverage over the underlying fluid retaining non-woven core 20. It has been found that such coverage may be achieved even at relatively low stitch densities resulting from long stitch lengths in the machine direction. Moreover, according to the potentially preferred form of the present invention wherein the bonding yarns are of a spun construction, surface coverage is enhanced still further by the fullness of such spun yarns. In addition, following formation, the contemplated construction of the textile composite 12 as described above is believed to be highly suitable to undergo mechanically induced shrinkage in the machine direction to still further enhance surface coverage while at the same time providing stabilization against shrinkage due to multiple industrial laundering operations during the useful life of the product.

As will be appreciated, an incontinence pad 10 or reusable diaper 17 must necessarily undergo numerous laundering operations during its useful life. Cumulative shrinkage over a number of such washings may lead to premature failure of the incontinence pad 10 or diaper 17 due to separation between the textile composite 12 and the underlying fluid barrier layer 14 since those materials may tend to shrink at different rates. In order to address this issue, the present invention contemplates that following formation, the textile composite 12 may be subjected to a mechanical shrinking operation prior to attachment to the underlying fluid barrier layer 14. As previously indicated, this mechanical shrinking operation may also enhance the bulk of the textile composite thereby both increasing the thickness of the textile composite 12 and pushing the bonding yarns 24 at the user contact surface 26 closer together so as to enhance surface coverage. In addition, it is believed that the mechanical compression of the textile composite 12 serves to build in a reserve of stored compressive energy which can efficiently offset the contracting tensioning forces associated with shrinkage introduced during multiple laundering operations. Compacting of the textile composite thus acts as a buffer against shape altering shrinkage.

According to one potentially preferred practice, it is contemplated that the desired mechanically induced shrinkage may be introduced by use of a bladeless cavity linear compaction apparatus of the type which is believed to be available from Micrex Corporation having a place of business in Walpole Mass., USA. By way of example only and not limitation, one such apparatus is believed to be illustrated and described in U.S. Pat. No. 3,869,768 to Walton et al. the contents of which are incorporated by reference as if fully set forth herein. Textile compaction equipment and practices are also illustrated and described in U.S. Pat. No. 4,717,329 to Packard et al. and U.S. Pat. No. 5,149,332 to Walton et al. both of which are incorporated by reference as if fully set forth herein. Of course it is to be understood that other textile compaction devices as may be known to those of skill in the art may also be utilized.

By way of example only and not limitation, according to a potentially preferred practice the textile composite 12 is compressed in the length dimension by an amount sufficient to effect resistance against subsequent laundering induced shrinkage but without introducing substantial undulations across the surface. In general, in order to achieve this result for the textile composites as described above, it has been determined that a mechanical shortening in the length dimension should be in the range of about 5 percent to about 20 percent and will more preferably be in the range of about 10 percent to about 20 percent and will most preferably be in the range of about 13 percent to about 17 percent. Of course it is contemplated that compression at other levels including levels above about 20 percent may also be utilized if desired.

Actual levels of compaction may be determined empirically and varied as desired for a given textile composite structure. By way of example only and not limitation, it has been found that a textile composite of 18 gauge stitchbonded construction as previously described with a stitch density in the machine direction of about 8 stitches per inch and which undergoes a mechanical shortening of about 15 percent in the length dimension exhibits less than about 2.5% shrinkage in the length dimension following 5 or more washing and tumble drying cycles for normal/cotton sturdy fabrics according to AATCC Test Method 135-1995 as will be well known to those of skill in the art.

Surprisingly, it has been found that by selection of the stitch density in the machine direction in combination with a selected degree of mechanical shortening, it may be possible to achieve an unexpectedly substantial increase in the thickness of the textile composite 12 while at the same time gaining the benefit of substantial dimensional stability under laundering conditions as outlined above. In particular, it has been found that a level of increased thickness may be achieved such that the resulting product takes on a bulkiness generally corresponding to that of the traditional quilted textile products which have been used in prior fluid retaining pads and diapers. Heretofore, such bulkiness has been difficult to achieve in stitch bonded constructions. As will be appreciated, the term “bulkiness” is meant to refer to the character of the material which is the inverse of density.

It has been found that enhanced bulkiness may be particularly great in constructions of the textile composite 12 incorporating lower stitch densities. In particular, in order to obtain enhanced thickness and corresponding bulkiness during compaction, stitch densities in the machine direction of about 12 stitches per inch or less and more preferably about 9 stitches per inch or less and most preferably about 4 to about 8 stitches per inch may be desired. Such low stitch densities in the machine direction have been found to give rise to percentage increases in thickness which are substantially greater than the corresponding percentage reduction in the length dimension. That is, the compaction of such structures actually results in an increase in bulkiness (i.e a reduction in overall density).

While the reason for the substantial increase in bulkiness is not fully understood, it is believed that the long stitch lengths corresponding to such low stitch densities provide sufficient internal relaxation to permit the intermediate non-woven batting core 20 to expand outwardly upon the introduction of longitudinal compressive force thereby increasing the void volume within the batting. By way of example only and not limitation, it has been found that a stitch bonded textile composite as previously described with a stitch density in the machine direction of about 8 stitches per inch and which undergoes a mechanical shortening of about 15 percent in the length dimension exhibits a change in thickness from about 1.5 mm before compaction to about 2.2 mm following compaction. As will be appreciated, this corresponds to approximately a 47 percent increase in thickness which is more than three times the percentage decrease in the length dimension.

As previously indicated, following mechanical shrinkage, the low stitch density surprisingly does not negatively impact shrink resistance during laundering. Accordingly, a full and thick textile composite structure may be obtained which is stable during laundering and which utilizes highly efficient stitch bonding techniques.

While the present invention has been illustrated and described in relation to particularly preferred embodiments and constructions, it is to be understood that such embodiments and constructions are illustrative only and the present invention is in no event to be limited thereto. Rather, it is contemplated that modifications and variations to the present invention will no doubt occur to those of skill in the art upon reading the above description and/or through practice of the invention. It is therefore contemplated and intended that the present invention shall extend to all such modifications and variations which incorporate the broad aspects of the present invention within the full spirit and scope thereof.

1. A method of forming a launderable fluid containment textile structure useful in absorbing fluid discharged by a user, wherein the textile structure is of stitch bonded construction having a machine direction and a cross-machine direction, the method comprising:
(a) providing a fluid retaining core layer of non-woven batting comprising a blend of hydrophobic textile fibers and hydrophilic textile fibers such that said hydrophobic textile fibers and hydrophilic textile fibers are dispersed substantially throughout said core layer;
(b) applying a plurality of bonding yarns in a repeating stitch bonding pattern through said core layer to form a preliminary stitch bonded composite having a technical face and a technical back and wherein segments of said bonding yarns define a user contact surface across the core layer; and
(c) mechanically shrinking the preliminary stitch bonded composite in at least the length dimension corresponding to the machine direction of the textile structure by introduction of mechanical compression, whereby the preliminary stitch bonded composite is shortened prior to use.

2. The method according to claim 1, wherein said bonding yarns are of a spun construction.

3. The method according to claim 2, wherein said bonding yarns comprise hydrophobic constituent fibers and hydrophilic constituent fibers.

4. The method according to claim 3, wherein at least a portion of said bonding yarns are open end spun yarns.

5. The method according to claim 3, wherein said hydrophobic constituent fibers comprise polyester fibers and said hydrophilic constituent fibers comprise cotton fibers.

6. The method according to claim 5, wherein said bonding yarns consist essentially of about 65% polyester and about 35% cotton.

7. The method according to claim 2, wherein said bonding yarns are characterized by a cotton count in the range of about 6 to about 36.

8. The method according to claim 2, wherein said bonding yarns are of a singles spun construction characterized by a cotton count in the range of about 6 to about 14.

9. The method according to claim 1, wherein said bonding yarns are textured polyester yarns.

10. The method according to claim 1, wherein in step “b” said bonding yarns are applied through said core layer at a stitch density in the machine direction of not greater than about 16 stitches per inch.

11. The method according to claim 1, wherein in step “b” said bonding yarns are applied through said core layer at a stitch density in the machine direction of not greater than about 12 stitches per inch.

12. The method according to claim 1, wherein in step “b” said bonding yarns are applied through said core layer at a stitch density in the machine direction of about 8 stitches per inch or less.

13. The method according to claim 1, wherein in step “b” said bonding yarns are applied through said core layer in a stitch bonding pattern such that said bonding yarns cooperatively define said user contact surface across the technical face of the preliminary stitch bonded composite.

14. The method according to claim 1, wherein in step “c” the preliminary stitch bonded composite is shortened by not less than about 5 percent in the length dimension.

15. The method according to claim 1, wherein in step “c” the preliminary stitch bonded composite is shortened by about 15 percent or more in the length dimension.

16. The method according to claim 1, wherein said bonding yarns include a cotton constituent and wherein the method comprises the further step of selectively overdying the user contact surface to a shade other than white.

17. A method of forming a launderable fluid containment textile structure useful in absorbing fluid discharged by a user, wherein the textile structure is of stitch bonded construction having a machine direction and a cross-machine direction, the method comprising:
(a) providing a fluid retaining core layer of non-woven batting comprising a blend of hydrophobic textile fibers and hydrophilic textile fibers such that said hydrophobic textile fibers and hydrophilic textile fibers are dispersed substantially throughout said core layer;
(b) applying a plurality of bonding yarns of spun construction including both hydrophobic and hydrophilic constituent fibers in a repeating stitch bonding pattern through said core layer at a stitch density in the machine direction in the range of about 6 to about 10 stitches per inch to form a preliminary stitch bonded composite having a technical face and a technical back and wherein segments of said bonding yarns define a user contact surface extending in contacting relation across the core layer such that there is no intermediate layer between the core layer and the user contact surface; and
(c) mechanically shrinking the preliminary stitch bonded composite in at least the length dimension corresponding to the machine direction of the textile structure by introduction of mechanical compression, such that the preliminary stitch bonded composite is shortened by about 15 percent or more prior to use without the introduction of undulations within the textile structure and such that the thickness of the preliminary stitch bonded composite is increased by more than about 30 percent whereby the overall bulkiness of the preliminary stitch bonded composite is increased.

18. The method according to claim 17, wherein said bonding yarns comprise polyester fibers in combination with substantially hydrophilic fibers selected from the group consisting of cotton fibers and rayon fibers.

19. The method according to claim 18, wherein said bonding yarns consist essentially of about 65 percent polyester and about 35 percent cotton.

20. The method according to claim 18, wherein said bonding yarns are of a singles open end spun construction characterized by a cotton count of about 10.

21. The method according to claim 17, wherein in step “b” said bonding yarns are applied through said core layer in a stitch bonding pattern such that said bonding yarns cooperatively define said user contact surface across the technical face of the preliminary stitch bonded composite.

22. The method according to claim 19, wherein said stitch bonding pattern is a chain stitch pattern.

23. An incontinence pad comprising a launderable fluid containment textile structure formed by the method as recited in claim 1.

24. An incontinence pad comprising a launderable fluid containment textile structure formed by the method as recited in claim 17.

25. A diaper comprising a launderable fluid containment textile structure formed by the method as recited in claim 1.

26. A diaper comprising a launderable fluid containment textile structure formed by the method as recited in claim 17.
